(19) United States
(12) Patent Application Publication
(10) Pub. No.: US 2026/0060739 A1
(43) Pub. Date: Mar. 5, 2026

(54) GENERATOR WITH CHARACTERISTIC CURVES SWITCHING

(71) Applicant: Erbe Elektromedizin GmbH, Tuebingen (DE)

(72) Inventors: Konstantin DORNHOF, Horb am Neckar (DE); Peter Selig, Nehren (DE)

(21) Appl. No.: 19/309,689

(22) Filed: Aug. 26, 2025

(30) Foreign Application Priority Data
Aug. 28, 2024 (EP) 24196861.9
Apr. 24, 2025 (EP) 25172292.2

(51) Int. Cl. A61B 18/12; H03K 17/56

(52) U.S. Cl. CPC A61B 18/1206; H03K 17/56

(57) ABSTRACT

A generator (11) for powering various instruments (35, 44) is described which provides the desired output characteristic curves without the aid of a control loop. For this purpose, the generator uses a reactive network (19) that has the desired characteristic curves by itself (i.e., intrinsically). This is achieved by providing various selectable complex resistances in the output branch of the generator (11), which provide different relationships between output power and load resistance.

### CROSS REFERENCE TO RELATED APPLICATIONS

This application claims the benefit of European Patent Application No. 24196861.9, filed Aug. 28, 2024, and European Patent Application No. 25172292.2, filed Apr. 24, 2025, which are incorporated herein by reference in their entireties.

### TECHNICAL FIELD

The invention relates to a generator for supplying an electrosurgical instrument with current to cause a tissue change, in particular a devitalizing tissue change. The invention also relates to a system comprising the said generator and a plurality of instruments connectable to the generator, which fulfill different tasks and for this purpose require different current/time curves, voltage/time curves, current/voltage characteristic curves or different relationships between tissue resistance and power introduced into the tissue (tissue resistance/power characteristic curves) on the part of the generator.

### BACKGROUND

Electrosurgical instruments such as electrosurgical scalpels, cauterization forceps or the like are known. Various generator concepts are known for powering such instruments.

US 2022/0313345 A1 discloses a generator with a resonant circuit which is excited by two transistor amplifiers operating in push-pull mode in a cascode circuit. The generator can be arranged completely or partially in the instrument. To supply power to the electrodes of the instrument, the resonant circuit is provided with a decoupling coil which, depending on the number of electrodes, can have two or three connections.

EP 2 499 982 A1 discloses a generator with a sensor circuit containing several sensors for detecting tissue and energy properties, such as tissue impedance, tissue temperature, current and/or voltage output. This sensor circuit provides a feedback signal to the generator controller. This feedback forms a control loop that regulates the current output of the generator powering the instrument as required.

A similar generator is known from EP 1 862 137 A1. This generator also uses a sensor circuit that detects the voltage and current at the generator output and then controls the generator accordingly. This is also the case with the generator according to EP 1 051 948 A2.

EP 2 520 241 B1 also provides a control loop for controlling the operation of the generator, whereby the control loop serves to establish a desired relationship between the current flowing through the tissue and the voltage applied, whereby these characteristic curves can be defined as linear or non-linear.

EP 2 405 842 B1 also discloses a generator with an output-side transformer, downstream of which a series resonant circuit is connected to adapt to a load to be connected. In one of the embodiments shown, the resonant circuit can be connected to various taps of the output-side transformer of the generator via switches.

Generators of this type should often be able to supply different types of instruments with surgical current. Cauterization instruments, for example, require fundamentally different values and time curves of voltage and current than, for example, electro scalpels, which in turn require different curves of voltage and current than, for example, ablation instruments or plasma probes. For this reason, such generators typically have a mode selector switch for selecting different modes (coagulation, cutting, cauterization, etc.), with which the generator controller is given different values and time curves for current, voltage or other electrical parameters (frequency, modulation, crest factor, power or limit value for one or more of these parameters).

Control loops are used to achieve the desired output-side behavior of the generators, but these are subject to design-related restrictions. For example, control oscillations can occur during rapid load changes, leading to significant temporary deviations between the desired current and the actual current flowing. However, if the voltage or current deviate significantly from the set value, even if only briefly, e.g., as part of a control oscillation, undesirable treatment effects can occur. For example, sticking effects can occur during coagulation. If an electrode sticks to the tissue, tearing can lead to unwanted lesions that can impair the surgical result. When cutting, too strong or too weak coagulation of the cutting edge can also cause bleeding or adhesive effects, which are undesirable.

### SUMMARY

Based on this, it is one object of the invention to specify an improved generator.

This object is achieved with a generator as described herein.

On the output side, the generator according to the invention has a reactive network comprising a plurality of inductors and a plurality of capacitors which can be connected or are connected to the generator output in optional pairings. Each pairing forms an output branch and is formed by the series connection of at least one of the inductors and a capacitor selected from a group of capacitors. Depending on which output branch of the output network is used, the output network of the generator determines different internal resistances of the generator, which lead to different output characteristic curves. This is preferably done without feeding back measured values of current and voltage to the clock generator or the controlled switch for energizing the resonant circuit. The relationship between the tissue resistance and the power applied to the tissue, which is necessary for the operation of a specific instrument, is then caused exclusively by the tissue resistance, which changes over time during the course of treatment. In other words, the internal impedance, i.e., the complex internal resistance of the generator and thus the output characteristic curve, are determined according to the mode in such a way that the desired surgical effect is achieved on the connected instrument without control intervention. Changing the tissue resistance results in a shift of the operating point on the output characteristic curve of the generator and thus in the desired adjustment of current and voltage. The various output branches can lead to different outputs of the generator or can be connected to a two-pole or multi-pole output of the generator via an optionally provided switch unit.

The different output branches enable the different modes (cutting, coagulation, etc.) to be selected quickly and easily and load changes or load currents during use do not lead to control oscillations and possibly undesirable surgical effects. The treatment current output by the generator results from the direct interaction of the generator internal resistance and the tissue resistance. For example, in one generator setting, coagulation and dissection instruments, such as those used for vessel sealing and vessel separation, can be supplied with current and voltage via the generator according to the invention without the need for control intervention to determine the voltage or current. The changing resistance of the treated tissue leads to a change in the voltage applied to the electrodes during the course of treatment, whereby the voltage changes correspond to the course of treatment by means of corresponding characteristic curves that correspond to the surgical process. In another generator setting, another instrument, e.g., an electro scalpel, can also be powered without control intervention.

The primary-side inductor is referred to below as the primary inductor. The primary-side capacitor is referred to below as the primary capacitor. The primary inductor and the primary capacitor form a parallel resonant circuit. This parallel resonant circuit is connected to one or more electronic switches, which are used to excite the parallel resonant circuit to oscillate. The (at least one) electronic switch is alternately opened and closed by a clock generator, whereby the switching signal output by the clock generator for this purpose can have a predetermined frequency. Alternatively, the resonant circuit can be part of a free-running oscillator circuit. The frequency can be time-invariant and therefore a fixed frequency. However, it is possible to design the clock generator in such a way that the clock signal is subject to modulation, for example pulse width modulation or frequency modulation. It can also be amplitude-modulated with a different frequency, for example on/off-sampled, whereby this modulation frequency can also be pulse-width modulated. The modulation of the clock generator can be set according to the selected operating mode. Again, the modulations of the various modes can be fixed.

The secondary-side inductors of the generator are referred to below as secondary inductors. The secondary inductors are closely coupled to the primary inductor. The coupling factor is preferably above 0.9, preferably above 0.95 and most preferably at or above 0.97. The number of windings of the inductors is based on the desired open-circuit voltage when the generator is idling. The number of windings of a secondary inductor is preferably less than the number of windings of the primary inductor. At least preferably, the sum of the number of windings of all secondary inductors is also at most as large as the number of windings of the primary inductor.

The secondary-side capacitors are referred to here as secondary capacitors. One or more secondary inductors are connected to an output pole of the generator via a circuit branch. The circuit branch contains a secondary capacitor and can contain a switching path of a selector switch (i.e., a switch unit) which is connected in series with the secondary capacitor. These series circuits can have the same or different resonant frequencies. Due to the strong coupling of the primary and secondary inductors, the secondary capacitors are transformed into the primary circuit and thus reduce its resonant frequency. Preferably, the switching frequency of the at least one switch used to excite the primary resonant circuit is higher than the resonant frequency of the resonant unit formed by the primary resonant circuit and the secondary capacitors. This applies to at least one or more modes, preferably to all of them.

In the generator according to the invention, the resonant frequency of the above-mentioned oscillating unit can change depending on the impedance of the energized tissue. This effect can be used to establish the desired relationship between the tissue resistance and the power introduced into the tissue.

By activating one of the series circuits mentioned (i.e., switching on the respective switching path) and deactivating the other series circuits (switching off the respective switching path), the output characteristic of the generator is significantly influenced. It is possible to give the various series circuits characteristic curves that enable different treatment modes. For example, the generator can be used for coagulation instruments as well as for dissection instruments and other instruments without having to generate the required output characteristic via a control loop. Instead, the respective output characteristic curve is provided solely by the reactive network, which consists of the primary-side resonant circuit and the series circuit respectively activated on the output side.

The selector switch can also be connected to the clock generator. Alternatively, a signal controlling the mode selector switch can be fed to the clock generator. In both cases, the clock generator can be configured to supply a clock signal adapted to the requirements of the selected mode.

### BRIEF DESCRIPTION OF THE DRAWINGS

Further details of advantageous embodiments of the invention are the subject of the drawing, the associated description or of claims. The figures show:

FIG. 1 an overview diagram of the generator according to the invention with connected instrument, in symbolic representation,

FIG. 2 the generator according to FIG. 1, in a further symbolic representation,

FIG. 3 the generator according to FIGS. 1 and 2 as an overview circuit diagram, connected to a first instrument,

FIG. 3a the generator shown in FIGS. 1 and 2 in push-pull circuit as an overview circuit diagram, connected to the instrument,

FIG. 3b a free-swinging embodiment of the generator embodiment shown in FIG. 3a,

FIG. 4 the generator according to FIG. 3 connected to another instrument,

FIG. 4a a modified generator for connecting the instrument shown in FIG. 4,

FIG. 4b the instrument shown in FIG. 4, connected to two generators as shown in FIGS. 3, 3a or 3b,

FIG. 4c the instrument shown in FIG. 4, connected to a generator with two different output circuits for two different pairs of electrodes of the instrument shown in FIG. 4,

FIG. 5 various output characteristic curves of the generator in the settings according to FIGS. 3 and 4,

FIG. 6 an equivalent circuit diagram to illustrate the function of the generator according to FIGS. 3 and 4,

FIG. 7 resonant frequencies of the generator resonant circuit at different settings,

FIG. 8 switching signal sequences of the generator's clock generator.

### DETAILED DESCRIPTION

FIG. 1 symbolically illustrates a load resistance 10 formed by a patient and an instrument and a generator 11 powering the load resistance 10. The load resistance 10 has a load impedance Z_{L} which is dependent on the tissue type and the type of treatment as well as the progress of the treatment, i.e., the elapsed time and the intensity of the current supply. The type of treatment influences the load impedance Z_{L} to the extent that the shape and size of the electrode, the intensity of the contact between the electrode and the tissue and the condition of the tissue (wet, dry, coagulated, etc.) play a role. The load impedance Z_{L} is therefore shown in FIG. 1 as a variable complex resistance.

The load resistance 10 is connected via two lines 12, 13 to two poles 14, 15 of a generator output 16. The generator 11 contains a high-frequency voltage source 17, whose complex internal resistance 18 is illustrated in FIG. 1 as a separate circuit symbol. The complex internal resistance 18 has an impedance Zᵢ, which can have a linear or non-linear current/voltage characteristic curve.

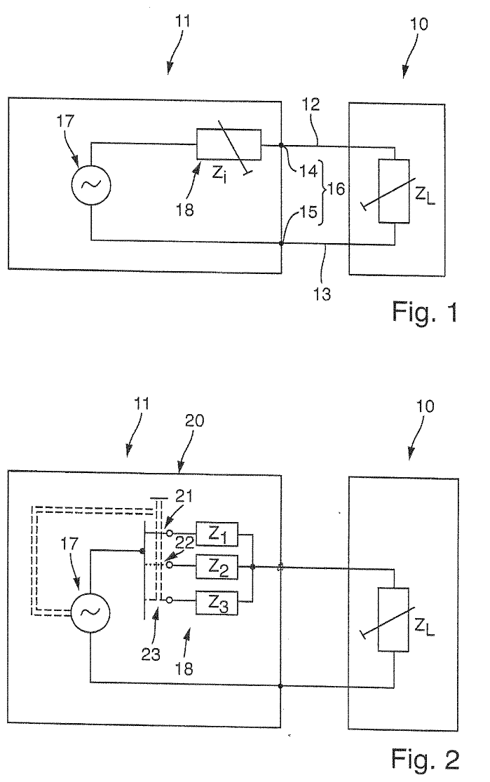

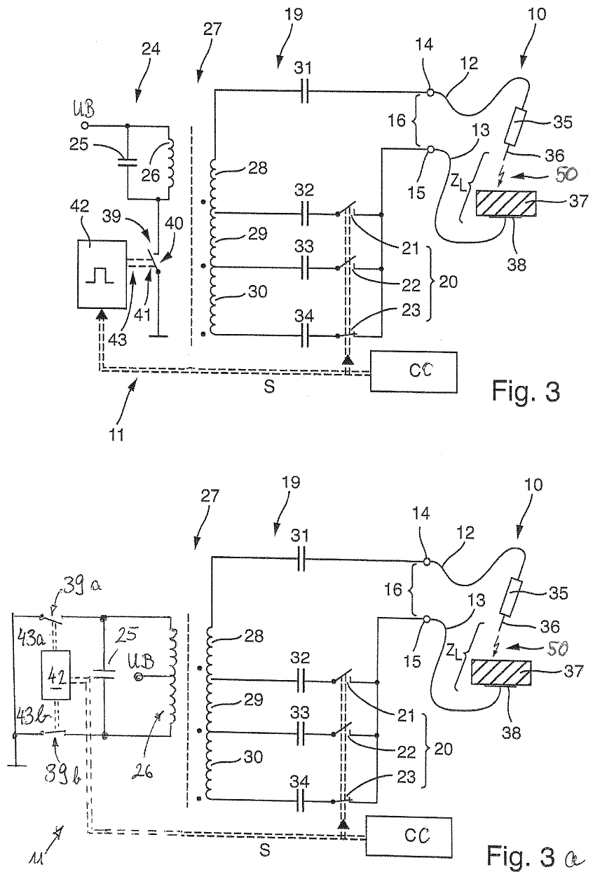

The internal resistance 18 can be varied in stages so that it can assume discrete different impedances Z₁, Z₂, Z₃, as is indicated in FIG. 2. The impedances Z₁, Z₂, Z₃ of the internal resistance 18 can be formed by different branches of a reactive network 19, which is illustrated in FIG. 3. A selector switch 20, which has different switching paths 21, 22, 23, is used to activate or deactivate the different branches. The selector switch 20 can be a manual switch or a switch controlled by a control signal S. A control module CC can be used to generate the control signal.

The number of switching paths 21 to 23 depends on the number of different impedances Z₁ to Z₃of the internal resistance 18 to be realized and thus the number of output characteristic curves and corresponding modes of the generator 11 to be realized. The selector switch 20 is designed in such a way that only one of its switching paths 21 to 23 can be electrically conductive (permeable), while all other switching paths are impermeable (blocked). The switches can be contactless electronic switches, mechanical switches with a switching contact or a socket arrangement that offers a choice of several poles for one of the lines 12, 13. A control signal S can be used to control the selector switch 20, which is provided by a manually operated switch or a generator controller not illustrated further.

As illustrated in FIG. 3, for example, the generator 11 includes a primary-side resonant circuit 24 comprising a primary capacitor 25 and a primary inductor 26 which are electrically connected in parallel to each other. The resonant circuit 24 is tuned to a resonant frequency of several 100 kHz, e.g., to a frequency of 480 kHz. For this purpose, the primary capacitor 25 can have a value of 2.2 nF and the primary inductor 26 a value of 50 μH. However, other values for the resonant frequency, the primary capacitor 25 and the primary inductor 26 are possible.

The primary inductor 26 is preferably formed by the primary winding of a high-frequency transformer 27. The high-frequency transformer 27 has a plurality of secondary windings which form secondary inductors 28, 29, 30 and couple inductively to the primary inductor 26. The secondary inductors 28, 29, 30 can have the same or different numbers of windings and thus the same or different inductance values. They can be wound as individual coils or formed by a single coil which has several taps and thus divides the coil into the individual secondary inductors 28, 29, 30. The number of secondary inductors 28, 29, 30 corresponds to the number of switching paths 21 to 23 and can vary according to the number of desired output characteristic curves of the generator 11 to be realized. The secondary inductors 28, 29, 30 preferably each have a number of windings which is less than the number of windings of the primary inductor 26. Further preferably, the sum of the numbers of windings of the secondary inductors 28, 29, 30 is not significantly greater, in the preferred case even at most as great as the number of windings of the primary inductor 26.

The first inductor 28 is connected to the pole 14 of the generator output 16. A coupling capacitor 31 can be arranged between the secondary inductor 28 and the generator output 16. This is optional in all embodiments of the generator 11 described here and below. The other end of the secondary inductor 28 is connected to the other pole 15 of the generator output 16 via a secondary capacitor 32 and the switching path 21 of the selector switch 20. The secondary inductor 28 forms a first output branch 28/32 with the secondary capacitor 32. The secondary inductor 28 and the secondary capacitor 21 form a first inductively powered series circuit for selectively powering the output 16. The sequence of the secondary capacitor 32 and the switching path 21 can be as shown in FIG. 3 or vice versa.

The winding ends of the secondary inductors 28, 29, 30 are each marked with a dot in FIG. 3. This is important for the following explanation of the interconnection of the secondary inductors 28 to 30.

The start of the winding of the secondary inductor 29 is connected to the end of the winding of the secondary inductor 28. Similarly, the start of the winding of the secondary inductor 30 is connected to the end of the winding of the secondary inductor 29. A circuit branch extends from the winding end of the secondary inductor 29, in which a further secondary capacitor 33 is arranged, which forms a series connection with the secondary inductor 29 and the secondary inductor 28. This series circuit forms a second output branch 29/33. This series circuit can be connected to the pole 15 of the generator output 16 via the switching path 22. The sequence of the secondary capacitor 33 and the switching path 22 can be as shown in FIG. 3 or vice versa.

The winding end of the secondary inductor 30 forms a series connection with a third secondary capacitor 34, which can be optionally connected to the pole 15 of the generator output 16 via the switching path 23 of the selector switch 20. This series connection forms a second output branch 30/34. The sequence of the secondary capacitor 34 and the switching path 23 can be as illustrated in FIG. 3 or vice versa. Further secondary inductors, secondary capacitors and switching paths can be provided in a similar manner.

The secondary inductors 28, 29, 30 can have the same or different values and couple inductively with the primary inductor 26. The coupling factor is preferably greater than 0.95, more preferably greater than 0.97. The secondary capacitors 32, 33, 34 have descendingly staggered values. The secondary capacitor 32 is larger than the secondary capacitor 33, which in turn is larger than the secondary capacitor 34. The coupling capacitor 31, if present, is preferably larger than all secondary capacitors. In particular, it can be larger than the sum of the capacitances of all secondary capacitors 32 to 34. This also applies if, contrary to what is shown, there are not only three different circuit branches with three switching paths 21 to 23 and therefore also several secondary inductors and several secondary capacitors. The number of windings of the secondary inductors 28, 29, 30 are matched to the number of windings of the primary inductor 26 so that the voltage at the secondary inductors 28-30 is at most as high as the voltage in the primary (parallel) resonant circuit 24. The inductors 26, 28, 29, 30 thus form a transformer that transforms down the resonant circuit voltage with factors of e.g., 1.5:1, 2:1, 3:1 or other ratios or decouples it at most 1:1. Conversely, this increases the tissue impedance (at least if the largest transmission factor of 1:1 is not used) and transforms it into the resonant circuit 24, thus reducing the damping of the resonant circuit 24 by the tissue impedance.

A surgical instrument 35 with an electrode 36 is connected to the generator output 16, which is designed here as a monopolar instrument and is used to act on biological tissue 37, for example to make an incision. The biological tissue 37 (for example in the form of a living patient) is connected to the generator output 16 via a neutral electrode 38. The instrument 35 with its electrode 36 and any spark gap 50 together with the biological tissue 37 and the contact resistance to the neutral electrode 38 form the load resistance 10. In general, the instrument 35 can be an instrument for open surgery, a laparoscopic instrument, an instrument for endoscopic use or a tool part that can be connected to an arm of a surgical robot.

An electronic circuit, which has at least one electronic switch 39, is used to excite the reactive network 19, in particular to excite the resonant circuit 24. This has a control path 40 and a control input 41, which is connected to a clock generator 42 in order to receive a control signal 43 from the latter. The clock generator 42 is preferably configured to emit the switching signal 43 as a square-wave signal with a fixed frequency. This frequency is preferably above 200 kHz and can, for example, be 350 kHz or even 480 kHz. Preferably, the clock frequency is below 5 MHz, more preferably below 1 MHZ

In the simplest embodiment, the switching signal 43 generated by the clock generator 42 is invariable for all selected operating modes, i.e., independent of the switching position of the selector switch 20. In the preferred case, however, not only a connection between the control module CC and the selector switch 20 is provided, but also a connection between the control module CC and the clock generator. This enables the clock generator 42 to output a suitable clock signal 43 for each selected operating mode (each mode). The clock signals 43 of the various modes can differ in their modulation. Preferably, these are square waves with a fixed frequency between 100 kHz and 5 MHZ, e.g., fixed 350 kHz or fixed 480 kHz. However, the frequency can also be fixed depending on the mode. Preferably, however, it is fixed at least within one mode.

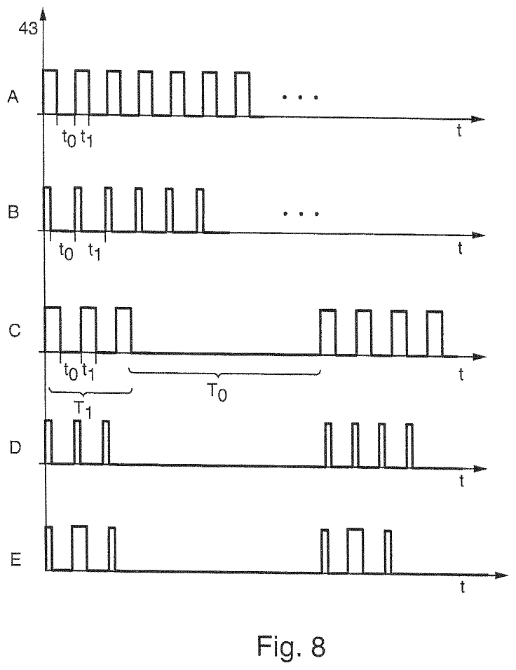

The clock signals 43 of the different modes can, for example, be unmodulated (“CW”—square wave continuous wave) with different pulse/pause ratios t₁/t₀ see FIG. 8.

The clock signal 43 can be defined according to the patterns A or B. The clock signals 43 can also be pulsed in groups with different group pulse/pause ratios T₁/T₀, see FIG. 8 clock signal 43 according to patterns C, D or F.

The control signal 43 can therefore be an uninterrupted pulse sequence (A, B) or a pulsed pulse sequence (C, D, F). In this case, the clock signal 43 is on/off-sampled, i.e., multiplied by a square-wave signal whose frequency is lower than the frequency of the control signal 43. This modulation frequency can be pulse-width modulated in order to make adjustments to different instrument requirements. In addition, the control signal 43 can also be pulse-width modulated, for example to fulfill power limitations or power specifications.

FIG. 3a illustrates a modified embodiment of the generator 11 with the instrument 35 connected thereto. The generator 11 is designed as a symmetrical push-pull oscillator whose switches 39a, 39b are opened and closed by the clock generator 42 in push-pull mode. The clock generator 42 can be designed to provide a fixed clock. Alternatively, the clock generator 42 can use the resonance of the parallel resonant circuit 24 to generate the control signals 43a, 43b. Such a clock generator and free-swinging oscillator are illustrated in FIG. 3b.

The oscillator 11 shown in FIG. 3b has, as switches 39a 39b, two transistors, preferably field-effect transistors or bipolar transistors, which are coupled together in the manner of an astable multivibrator. The base or gate of each transistor is connected, via a capacitor, to the collector or drain of the respective other transistor. Further transistors are connected in a base or gate circuit to these bipolar or field-effect transistors Ta, Tb, the collectors or drains of which are connected to the resonant circuit 24. The transistors 39a, Ta and 39b, Tb each form cascode circuits. A cascode circuit is the arrangement of two transistors in the signal flow path, of which the first transistor is operated in an emitter or source circuit and the subsequent transistor in the signal flow path is operated in a base or gate circuit.

As illustrated in FIG. 3b, the bases or gates of the transistors Ta, Tb are connected to a bias voltage UV, which, for example, can be derived from the operating voltage UB via a resistor RV and a Z diode DZ. If the bias voltage UV is constant, the transistors Ta, Tb operate with constant amplification and the generator 11 oscillates continuously. However, it is also possible to modulate the high-frequency oscillation produced by the generator in the resonant circuit 24. For this purpose, an electronic switch SW can be provided, which is arranged in a circuit branch connected in parallel with the Z diode DZ and which is controlled by the control signal S, e.g., clocked on/off. This clocking can take place with a frequency of a few kilohertz up to several 10 kHz. The control signal S can also be used to provide the high-frequency output voltage of the generator 11 with a pulse width modulation.

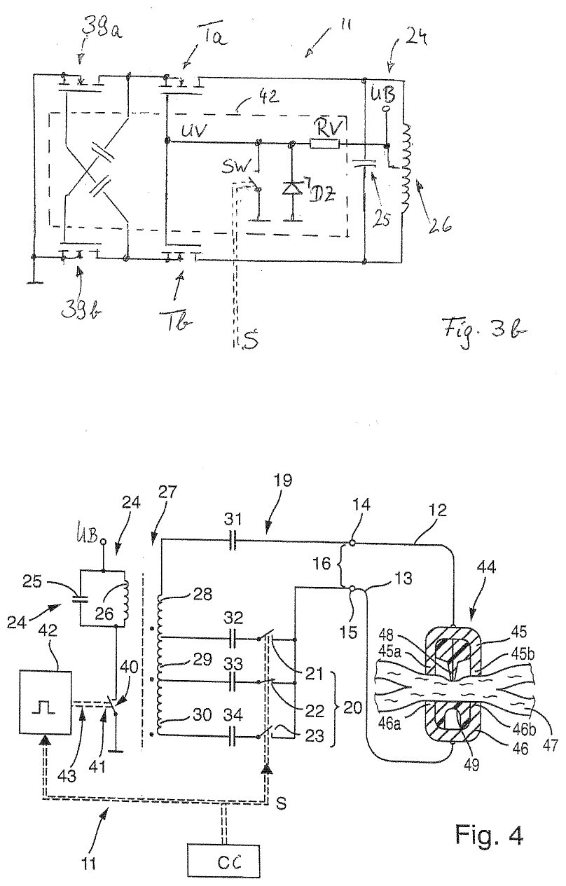

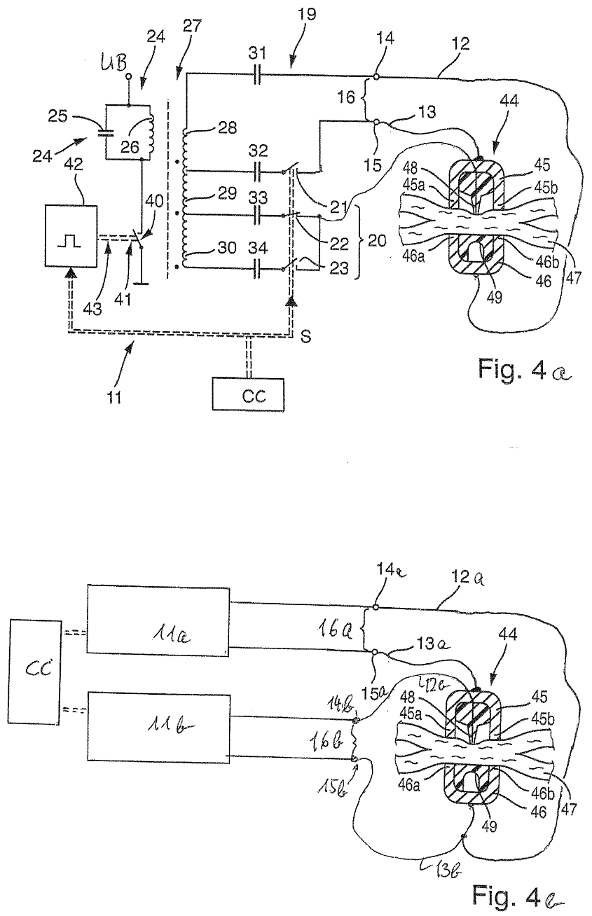

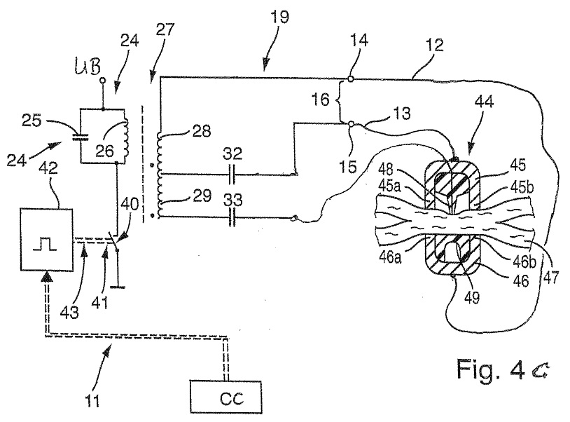

The generator 11 according to FIG. 3 is illustrated in FIG. 4, whereby another instrument 44 is connected to this generator 11, which is designed as a fusion and dissection instrument, for example. Such instruments are commonly used as bipolar instruments, for example for sealing and separating vessels, such as blood vessels. The instrument 44 can be an instrument for open surgery, a laparoscopic instrument, an instrument for endoscopic use or a tool part that can be connected to an arm of a surgical robot.

The instrument 44 includes two branches 45, 46, between which biological tissue 47, for example in the form of a blood vessel or another vessel, can be gripped. The branches 45, 46 each have two laterally spaced partial electrodes 45a, 45b, 46a, 46b, which serve as coagulation and fusion electrodes and which are connected to the lines 12, 13 for this purpose. A cutting electrode 48 which can be electrically connected to the branch 45 can be arranged between the partial electrodes 45a, 45b. Alternatively, the instrument 44 can include a voltage conversion device, for example a transformer, which is powered via the lines 12, 13 and supplies current to the cutting electrode 48.

Between the partial electrodes 46a, 46b, a preferably elastically formed abutment 49 can be arranged which presses the tissue 47 against the cutting electrode 48.

The instrument 44 shown in FIG. 4 can also be connected to the generator 11 shown in FIG. 3a or 3b and can be operated by it.

The generator 11 described in this respect operates in conjunction with the various instruments 35, 44 as follows:

When the instrument 35 is in operation, the control module CC sends a switching signal S to the selector switch 20 so that the switching path 23 is enabled while the switching paths 21, 22 are disabled. The secondary inductors 28, 29, 30 are thus connected in series with the secondary capacitor 34 from the point of view of the output 16. The coupling capacitor 31 completes the circuit. On the primary side, the electronic switch 39 is alternately opened and closed at a predetermined clock frequency f. The clock generator 42 outputs the switching signal 43 that is suitable for the selected mode. This is, for example, the switching signal with the pattern A in FIG. 8.

The frequency f of the switching signal 43 is preferably close to the resonant frequency of the resonant circuit 24. The secondary inductors 28, 29, 30 and the secondary capacitor 34 as well as the load impedance Z*_{L} transform into the primary side with the transformation ratio of the transformer 27.

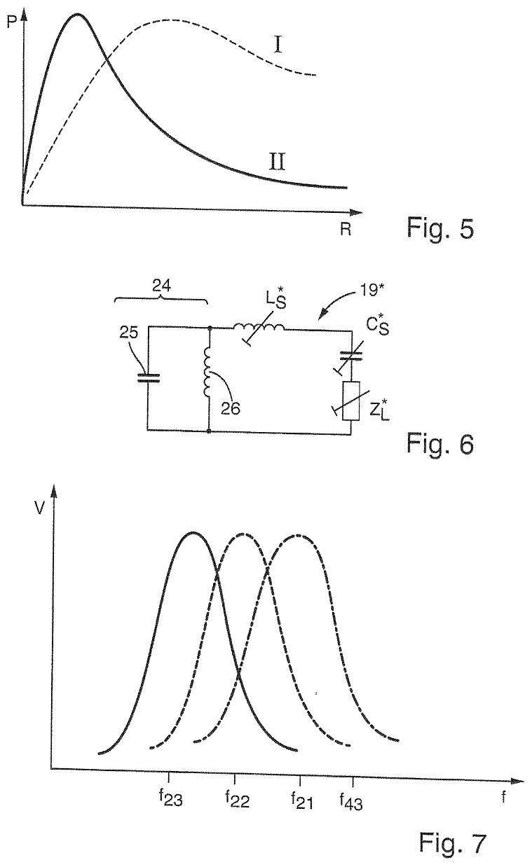

The equivalent circuit diagram is illustrated in FIG. 6. The overall result is a reactive transformed network 19*, whose characteristic curve I is illustrated in FIG. 5. The abscissa shows the value R of the load impedance Z*_{L} while the ordinate P shows the electrical power (apparent power) converted at the tissue resistance. The tissue resistance is low when the tissue is still damp. The power converted at the tissue is therefore still low, but increases sharply as the tissue dries out, reaching its maximum at medium tissue resistances. This means that 36 sparks can be maintained at the electrode, which lead to tissue cutting.

If, on the other hand, a bipolar coagulation and dissection instrument is to be connected to the generator output 16, the corresponding mode must be selected. For this purpose, the control module CC outputs a control signal S, as a result of which the selector switch 20 blocks the switching path 23 and instead releases the switching path 21 or, as shown in FIG. 4, the switching path 22. The lower inductance of the only two secondary inductors 28, 29 in conjunction with the higher capacitance of the secondary capacitor 33 now leads to a modified output characteristic curve II as shown in FIG. 5. In addition, the signal S simultaneously transmitted to the clock generator 42 can cause it to emit a different control signal 43, e.g., the control signal according to the pattern C in FIG. 8.

The maximum power introduced into the tissue is now already reached at lower tissue resistances, which leads to coagulation of the tissue between the partial electrodes 45a, 46a or 45b, 46b. In this state, the small-area cutting electrode 48 only transfers a small amount of current. As the tissue dries out, the power introduced into the tissue decreases. On the other hand, there is a current concentration at the cutting electrode 48, so that a cut can now be made despite the lower power.

As the schematic representation according to FIG. 7 shows, the activation of the various switching paths 21, 22, 23 can lead to a shift in the resonant frequency f of the reactive network 19 or transformed reactive network 19*. All three resonant frequencies f21, f22, f23 assigned to the switching paths 21, 22, 23 can be below the frequency f43 of the switching signal 43. In principle, however, it is also possible to set one or more of the frequencies F21, F22, F23 above the switching signal 43.

In all the embodiments described above, it was assumed that the switch 40 is opened and closed at a fixed predetermined frequency in order to excite the resonant circuit 24 to oscillate. In all embodiments, however, it is also possible to make the resonant circuit oscillate at its natural resonance by deriving the control signal 43 for the switch 40 from the oscillation frequency of the resonant circuit 24. The resonant circuit 24 is then the frequency-determining element of the oscillator circuit formed in this way. This applies in particular to the generator 11 shown in FIG. 3b. In a first variant, in the generator according to FIG. 3a, it is possible to use the control signal S to specify the switching frequency of the switches 39a, 39b and thus the oscillating frequency of the resonant circuit 24. In a second variant, the oscillator 11 of FIG. 3a can operate according to the principle of FIG. 3b and thus be free-running (self-controlled).

FIG. 4a shows the operation of the instrument 44 on the generator 11 with separate supply of the electrodes 45, 46, 48. The selector switch 20 can comprise two, as shown three, or also several switching paths 21, 22, 23 etc. The electrode pair 46/45 can then be connected to one of a plurality of available switching paths. Similarly, the electrode pair 46/48 can then be connected to one or one of a plurality of available switching paths. Thereby, it is possible to simultaneously or successively, with or without a time overlap, close and open the switching paths for supplying the electrodes 56, 45, 48. The electrode pairs 46/45, 46/48 can thus be operated serially or simultaneously, if necessary, whereby the electrode pairs 46/45, 46/48 are connected to different reactive networks. The generator 11 thus has different characteristic curves for the electrode pairs 46/45, 46/48.

The oscillator circuit connected to the resonant circuit 24 can be designed in the generator according to FIG. 4a according to the model of FIG. 3, as well as according to FIG. 3a or 3b.

FIG. 4b shows a further modification of the generator 11. According to this, the electrode pairs 46/45 of the instrument 44 are connected to a first generator 11a and the electrode pairs 46/48 are connected to a second generator 11b. Lines 12a, 13a; 12b, 13b can be used for this purpose. The generators 11a, 11b can each be designed as shown in FIGS. 3, 3a or 3b. They can also be connected to a common control module CC, which controls the generators 11a, 11b according to one of the principles described in connection with the generators of FIGS. 3, 3a, 3b, 4 or 4a.

FIG. 4c illustrates a further variant of the generator 11 according to the invention. The previous description of the generator according to FIG. 4b applies accordingly to this generator. In addition, the generators 11a and 11b are combined to form a common generator circuit. With regard to the transformer 27 on to the primary side, the generator 11 follows the model of the generators 11 shown in FIGS. 3, 3a, 3b 4 or 4a. With regard to transformer 27 on to the secondary side, the generator follows the model of the generators shown in FIG. 4a or 4b. The special feature of the generator 11 shown in FIG. 4c is that the switch unit 20 is completely missing. It is also possible to provide a switch unit with switching paths 21, 22 (not shown), whereby these are permanently closed (i.e., conductive) at least during the energization of the electrode pairs 46/45 and 46/48 at the same time.

In all embodiments, the generator can be arranged completely or partially in the instrument 35, 44. In particular, the clock generator 42, the resonant circuit 24, the transformer 27 as well as the coupling capacitor 31 and the capacitors 32, 33, 34 and the selector switch 20 can be part of the instrument 35, 44. The power supply device for providing the operating voltage UB, which is not illustrated further, can be arranged in an external device which is connected to the generator via an electrical line. The control module CC can be part of the separate device. Alternatively, it can be integrated into the generator 11 and arranged with it in the instrument 35, 44. Alternatively, the generator 11 of any type described herein can also be arranged completely in the separate device. Furthermore, it applies to all embodiments of the generator 11 that different sockets connected to the individual output branches 28/32, 29/33, 30/34 for connecting different instruments can be provided instead of the switch unit 20.

The concept according to the invention proposes a generator 11 for powering various instruments 35, 44, which provides the desired output characteristic curves without the aid of a control loop. For this purpose, it uses a reactive network 19 that has the desired characteristic curves by itself (i.e., intrinsically). This is achieved by providing complex resistances in the output branch of the generator 11 that can be switched on as required and that provide different relationships between the power output and the load resistance.

### REFERENCE SIGNS

10 load resistance
11 generator
Z_{L} load impedance
R amount of the load impedance Z_{L}
12, 13 lines
14, 15 pole
16 generator output
17 high-frequency voltage source
18 complex internal resistance
Zᵢ impedance of the internal resistance 18
Z₁, Z₂, Z₃ impedances of the internal resistance 18
19 reactive network
19* transformed reactive network
20 selector switch
21-23 switching paths of the selector switch
S control signal
24 resonant circuit
25 primary capacitor
26 primary inductor
27 high-frequency transformer
28, 30 secondary inductors
31 coupling capacitor
32, 34 secondary capacitors
35 instrument
36 electrode
37 biological tissue
38 neutral electrode
39 switches
40 control section
41 control input
42 clock generator
43 control signal
44 instrument
45,46 branches
45a, 45b partial electrodes of branch 45
46a, 46b partial electrodes of branch 46
47 tissue
48 cutting electrode
49 abutment
50 spark gap
Z*_{L} transformed load impedance
L*_{S} transformed secondary inductance
C*_{S} transformed secondary capacitance

1. A generator (11) for supplying an electrosurgical instrument (35, 44) with a current which causes a tissue change, the generator comprising:
a resonant circuit (24) with a resonant frequency (f_{R}), the resonant circuit (24) comprising a primary-side inductor (26) and a primary-side capacitor (25) connected in parallel thereto;
at least one electronic switch (39) comprising a control path (40) connected to the resonant circuit (24) and a control input (41) via which the control path (40) can be switched alternately between a conducting and a non-conducting state;
with a clock generator (42) configured to generate a switching signal (43) and is connected to the control input (41) to switch the control path (40) on and off;
a first and a second secondary-side inductor (28, 29) inductively coupled to the primary-side inductor (26);
a first secondary-side capacitor (32) having a first series connection with the first secondary-side inductor (28); and with
a second secondary-side capacitor (33) having a second series connection with the second secondary-side inductor (29); and
a selector switch (20) comprising two switching paths (21, 22), wherein one of the two switching paths is connected in series with the first secondary-side inductor (28) and the first secondary-side capacitor (32), and another of the two switching paths is connected in series with the second secondary-side inductor (29) and the second secondary-side capacitor (33).

2. The generator according to claim 1, wherein the first secondary-side inductor (28) and the first secondary-side capacitor (32) are connected in series via a switching path (21) of a selector switch (20) to one electrode (38 or 36) of a patient-side electrode pair (36/38, 46/45, 46/48), wherein the first secondary-side inductor (28) is connected at an end thereof opposite the secondary-side capacitor (32) to another electrode (36 or 38) of the patient-side electrode pair (36/38, 46/45, 46/48).

3. The generator according to claim 1, further comprising a third secondary-side inductor(30) inductively coupled to the primary-side inductor (26), the third secondary-side inductor having a third series connection with a third secondary-side capacitor (34); and
wherein the selector switch (20) is configured to connect at any given time only one of the first, second and third series connections of one of the secondary-side inductors (28, 29, 30) and one of the secondary-side capacitors (32, 33, 34) to an electrode of a patient-side electrode pair (36/38, 46/45, 46/48).

4. The generator according to claim 1, wherein the clock generator (42) is configured to generate a control signal (43) at a predetermined frequency.

5. The generator according to claim 4, wherein the clock generator (42) is configured to generate the control signal (43) in accordance with the resonant frequency of the resonant circuit (24).

6. The generator according to claim 1, wherein the generator (11) has different internal resistances (Z1, Z2, Z3) corresponding to different settings of the selector switch (20).

7. The generator according to claim1, wherein each of the secondary-side capacitors (32, 33, 34) and each of the secondary-side inductors (28, 29, 30) are dimensioned such that the resonant frequency of the parallel resonant circuit is greater than 200 kHz.

8. The generator according to claim 1, wherein the clock generator (42) is configured to output the control signal (43) at a switching frequency (f) that is greater than the resonant frequency (f_{R}) of the resonant circuit (24).

9. The generator according to claim 1, wherein each of the secondary-side inductors (28, 29, 30) are connected in series with one another.

10. The generator according to claim 1, wherein the secondary-side capacitors (32, 33, 34) each have different capacitance values.

11. The generator according to claim 1, wherein a capacitance value of each of the secondary-side capacitors (32, 33, 34) is smaller than a capacitance value of the secondary-side inductor (28, 29, 30) connected in series therewith P

12. The generator according to claim 1, wherein the generator (11) is a push-pull oscillator.

13. The generator according to claim 1, further comprising an electrosurgical instrument (35) configured for coupling with an operating robot.

14. A system, comprising:
the generator (11) according to claim 1; and
an electrosurgical instrument (35, 44) comprising at least one pair of electrodes (36, 38) supplied by the generator (11).

15. The system according to claim 14, wherein the generator (11) is at least partially integrated into the electrosurgical instrument (35, 44).
